(19) United States
(12) Patent Application Publication
(10) Pub. No.: US 2025/0146995 A1
(43) Pub. Date: May 8, 2025

(54) PROCESS AND DEVICE FOR OPERATING A SENSOR ARRANGEMENT

(71) Applicant: Dräger Safety AG & Co. KGaA, Lübeck (DE)

(72) Inventors: Christof RODEHORST, Lübeck (DE); Jun FURUTA, Lübeck (DE); Kai EINECKE, Lübeck (DE)

(21) Appl. No.: 18/937,198

(22) Filed: Nov. 5, 2024

(30) Foreign Application Priority Data
Nov. 7, 2023 (DE) 10 2023 130 701.0

(51) Int. Cl. G01N 33/00; G06T 7/73

(52) U.S. Cl. CPC G01N 33/0075; G01N 33/0063; G06T 7/74

(57) ABSTRACT

An operating arrangement operates a sensor arrangement that includes at least one sensor (1.2). An installation phase is carried out for each sensor. An installation device generates at least one reference image of an installation location (Io.2) where the sensor is installed. A data set for the sensor is created in a central database, which includes each reference image. A subsequent search phase is carried out for at least one sensor. A search device (4) generates at least one search image (31.1, . . . , 31.4) of the surroundings of the search device. Each search image is compared with each stored reference image. The result of the image comparison is transmitted to the search device and is used by the search device to determine information (41.2) about the installation location of the sensor. The search device outputs the installation location information in a form that can be perceived by a human.

### CROSS REFERENCE TO RELATED APPLICATIONS

This application claims the benefit of priority under 35 U.S.C. § 119 of German Application 10 2023 130 701.0, filed Nov. 7, 2023, the entire contents of which are incorporated herein by reference.

### TECHNICAL FIELD

The present invention pertains to an operating process and an operating arrangement for operating a sensor arrangement. This sensor arrangement comprises at least one sensor, preferably several sensors. Furthermore, the invention relates to a system with such a sensor arrangement and such an operating arrangement.

### BACKGROUND

One example of such a sensor is a gas measuring device. In one application, a gas measuring device is able to measure the concentration of a target gas that is harmful or necessary for humans and, in one embodiment, to generate an alarm if the target gas concentration is above or below a predetermined concentration threshold. The target gas is in particular a combustible or toxic gas, but can also be oxygen, carbon dioxide or an anesthetic, for example.

Two possible ways of using such a gas measuring device are as follows:
Mobile (portable) gas measuring device: A user carries the gas measuring device with him/her while he/she is in an area where a target gas occurs or at least may occur. The gas measuring device outputs the target gas concentration and/or warns the user if it measures a target gas concentration above or below (oxygen) the concentration threshold.
Stationary (fixed) gas measuring device: The gas measuring device is installed at a location where at least one target gas occurs or at least can occur, and remains at this location during use. The stationary gas measuring device transmits alarms and/or measured target gas concentrations to at least one remote receiver.

This distinction between mobile and stationary also applies to other sensors.

The invention relates to a sensor arrangement with stationary sensors, so in the case of gas measuring devices as the sensors, it relates to the second application. Most of the time, no human is needed to operate a stationary sensor. However, in many cases a service technician must regularly check the sensor and if necessary calibrate and/or repair it.

### SUMMARY

It is an object of the invention to provide an operating process and an operating arrangement for operating a sensor arrangement with at least one stationary sensor, preferably with several stationary sensors, wherein the operating process and the operating arrangement should require less effort than known operating processes and operating arrangements.

The problem is solved by an operating process with features according to the invention and by an operating arrangement with features according to the invention. Advantageous embodiments of the operating process according to the invention are, where appropriate, also advantageous embodiments of the operating arrangement according to the invention and vice versa.

The operating process according to the invention and the operating arrangement according to the invention make it possible to operate a sensor arrangement. The sensor arrangement comprises at least one sensor, preferably several sensors. In a first alternative, the or each sensor of the sensor arrangement is capable of measuring at least one physical variable, optionally several variables. When it is referred to below that a sensor measures a physical variable, the following is meant: The sensor measures at least once which value this physical variable or another physical variable assumes at the respective time of measurement. The other physical quantity correlates with the physical quantity to be measured, i.e. it is an indicator for the quantity to be measured. In a second alternative, the or each sensor is capable of automatically deciding whether the respective physical quantity lies within a predetermined value range or does not. Preferably, this means the following: The sensor decides whether the value of the quantity is continuously within the value range or at least temporarily outside the value range in a reference period, the reference period being in particular a sliding time window or time point.

It is possible that the same sensor can both measure the value and decide whether the value falls within the specified value range or not. The alternatives can also be combined as follows: At least one first sensor is capable of measuring the physical quantity, and at least one second sensor is capable of automatically deciding whether the quantity falls within a predetermined value range or not.

The measured physical quantity can be the same for each sensor of the sensor arrangement. It is also possible that the sensors of the sensor arrangement are able to measure at least two different physical quantities in total. It is also possible that at least one sensor of the sensor arrangement is able to measure at least two different physical quantities simultaneously.

In the case of gas measuring devices as the sensors, each sensor is capable of detecting at least one predetermined target gas and/or measuring the concentration of at least one target gas. The fact that the target gas is predetermined means the following: The target gas or gases for which a spatial area is to be monitored is/are specified. The target gas is, in particular, a combustible or toxic gas or a gas that is otherwise harmful to humans or oxygen or another vital gas or carbon dioxide or an anesthetic. The target gas concentration sought is therefore the physical quantity to be measured, and a permissible range for the target gas concentration is specified as a value range. The target gas to be detected can be the same for each gas measuring device in the sensor arrangement. It is also possible that the sensors of the sensor arrangement are able to detect at least two different target gases in total and preferably distinguish them from each other. It is possible that at least one sensor of the sensor arrangement is able to measure a summed concentration of several simultaneously occurring target gases as the target gas concentration. As a rule, the physical quantity for which the sensor measures a value is a variable that correlates with the target gas concentration, in particular an electrical voltage or current or power or charge or temperature.

Each sensor of the sensor arrangement is installed at one installation location during a period of use, i.e. it is a stationary sensor in the sense of the distinction made at the beginning. It is possible that one human can carry the stationary sensor to the installation location and later transport it away from there again. As a rule, one human mounts (installs) each sensor at the respective installation location and puts the sensor into operation there. It is also possible for the stationary sensor to be placed on a surface for use. This human is called “the installation technician”. Of course, it is possible that several installation technicians are involved in the process of installing the sensors of the sensor arrangement. The process of installing the or each sensor of the sensor arrangement at the respective installation location is referred to as “the installation phase”.

When using the sensor arrangement, it is generally necessary or at least advisable for a human to check, maintain, calibrate and/or repair each sensor at the respective installation location during use. This human is referred to below as “the service technician”. The service technician usually has to locate (find) the sensor at the installation location at least once for this purpose, namely if maintenance is not possible exclusively from remote (from a distance), for example because a part of the sensor needs to be replaced, or if there is no data connection with the sensor. The latter is usually an undesirable situation that usually needs to be rectified on site. The sensor that needs to be calibrated or serviced or repaired is referred to below as a “sensor to be located” (a senor to be found). The process of locating (finding) at least one sensor of the sensor arrangement at its respective installation location is referred to as “the search phase”. Of course, it is possible that different service technicians are assigned to the sensors of the sensor arrangement. In practice, the situation often arises where the or at least one installation technician and the or at least one service technician are two different persons.

The operating arrangement according to the invention comprises a mobile data-processing installation device, a mobile data-processing search device (mobile locator), a central computer and a central database. The operating process according to the invention is carried out using such an operating arrangement.

A data-processing device is a device that is capable of processing data and/or signals and usually comprises a processor, an input unit and an output unit. The term “mobile device” refers to a device that is configured to be carried by a human and in general comprises its own power supply unit. In particular, a smartphone, a tablet or another portable computer are each a mobile and data-processing device within the meaning of the invention. It is possible for the same device to function both as the installation device and as the search device. In this implementation, two different programs are preferably installed on this device, namely a program for the steps of the installation phase and a program for the steps of the search phase. It is also possible for two different devices to function as the installation device and the search device respectively. These two devices may be of the same or different configuration and preferably use two different programs for the two different phases.

As a rule, the installation technician uses the installation device, and the service technician uses the search device. It is of course possible for different installation technicians to use the same installation device and/or different service technicians to successively use the same search device. It is also possible that the same human first works as an installation technician and uses the installation device and then the same human works as a service technician and uses the same or a different device as the search device. It is also possible that at least two different installation devices are used in the installation phase and/or at least two search devices are used in the or a search phase. In the following, we will refer to “the installation device” and “the search device” for short.

The central computer and the central database are generally each configured as a stationary device, but can also be implemented on a portable computer, for example on a smartphone. In particular, it is possible that the central computer and the central database are implemented on the installation device and/or on the search device. The central computer and the central database can also be implemented as components of a server on the Internet, in particular “in the cloud”. It is possible that a range of different servers is used in succession to implement the central computer and the central database.

A data connection is established at least temporarily from the installation device to the central computer. This data connection is preferably a wireless data connection, i.e. a data connection via radio waves, in particular using a public mobile radio network or a radio relay network. The data connection can also be a wired data connection. A unidirectional data connection is sufficient; a bidirectional data connection is also possible.

The installation device comprises an image recording device (image capture device/imaging device), in particular a digital camera or a video camera. The search device preferably also comprises an image recording device. The or each image recording device is capable of generating at least one image, preferably a sequence of images.

The operating process comprises an installation phase and a subsequent search phase. The installation phase is carried out for each sensor of the sensor arrangement. During the installation phase, the installation technician uses the installation device. The search phase is carried out for at least one sensor of the sensor arrangement wherein the sensor is to be located, i.e. when the service technician must or should locate the sensor in order to perform an action on this sensor. During the search phase, the service technician uses the search device. It is possible that a search phase is carried out for several or even all sensors of the sensor arrangement. It is also possible that several search phases are carried out in succession for at least one sensor each, even by different service technicians and/or with different search devices and also several times for the same sensor.

The installation device comprises an image recording device, preferably in the form of a digital camera. If the same mobile device is used both as the installation device and as the search device, the same image recording device of this mobile device is preferably used both in the installation phase and in the search phase.

As already mentioned, the installation phase is carried out for each sensor. The installation phase comprises the following steps for each sensor:
The image recording device of the installation device generates at least one reference image of the sensor's installation location. Preferably, the or at least one reference image shows the sensor installed at the installation location. It is possible that at least one further reference image shows an object in the vicinity of the sensor, in particular an object that obscures a view onto the sensor permanently or at least in one of several possible states. The object may be a door, for example. As a rule, the installation technician uses the image recording device of the installation device to generate the reference images.
The reference image or at least one, preferably each, reference image that was generated when the sensor was installed at the installation location is transmitted from the installation device to the central computer. The data connection described above is used for this purpose.
The central computer performs the following step: A data set (data record) for the sensor is created in the central database. If such a data set has already been created, this data set is supplemented as described below.
After creation or supplementation (completion), the data set comprises the or at least one, preferably each reference image that was taken at the installation location and transmitted to the central computer, optionally with a time stamp and/or with the respective geoposition (geolocation) for each reference image. The geoposition preferably describes the location at which the installation device recorded the reference image. In one implementation, a unique identifier of the sensor is captured (recorded/acquired/detected) at the installation location and transmitted to the central computer, and the data set comprises the captured and transmitted unique identifier. For example, the unique identifier can be captured by reading a reference image from the sensor. Or a reader, e.g. an NFC reader or barcode reader, reads the unique identifier of the sensor.

The or each search phase is carried out for at least one sensor to be located. The search phase comprises the following steps for the or each sensor to be located:
The data set for the sensor to be located in the central database is determined. The or each reference image covered (comprised) by the determined data set is determined.
The operating arrangement determines information about the installation location of the sensor to be located. For this determination, the operating arrangement uses the or each reference image determined, optionally further information.
The search device outputs the determined installation location information in at least one form that can be perceived by a human. In one embodiment, the output installation location information comprises the or at least one determined reference image of the sensor to be located.

In one embodiment, the search device itself determines the installation location information for a sensor. In another embodiment, a spatially remote data processing device determines the installation location information and this information is transmitted to the search device.

The inventors have internally identified the following problem: The service technician is often not exactly familiar with a plant or other site on which the sensor to be located is used. In many cases, the service technician who is supposed to maintain or repair a sensor is not the human who installed the sensor. A relatively long time often passes before a stationary sensor is located, for example, on a production plant or in a warehouse or storage hall or in a building or on board of a vehicle. It may be relatively difficult to locate the sensor at the installation location. In many cases, the installation location where the sensor is installed is also not documented in sufficient detail. Or the documentation is not available during the search phase or is outdated or not suitable to be transported by a service technician. The invention often provides a solution to this problem.

As already mentioned, in many cases the service technician is not also the installation technician. This is particularly the case if a technician from the sensor manufacturer installs the sensors as the installation technician and a technician from an operator of the sensor arrangement is later required to locate a sensor as the service technician. Therefore, the service technician often needs assistance in order to be guided to the installation location. Often, descriptions or other documents about the installation location are not available, outdated or not precise enough. The invention can be used in combination with such documents, but does not require that a document about the installation location is available or is made available.

The invention thus saves the time required for a service technician to locate a sensor. In many cases, thanks to the invention, the service technician has more productive time to maintain or repair the sensor or perform other work on the sensor, or can locate more sensors in the same time or requires less time. The invention also reduces the risk of a service technician not locating (finding) a sensor at all, or only locating it after a long search, or locating the wrong sensor.

The invention can be used in combination with a geoposition sensor, wherein the geoposition sensor is part of the search device and the search device uses measured geopositions to guide the service technician to the geoposition of the installation location of the sensor. Preferably, the data set for a sensor of the sensor arrangement includes the geoposition of the installation location, whereby this geoposition has been measured during the installation phase and stored as part of the data set for the sensor. Geoposition sensors as components of smartphones and other mobile data-processing devices are well known and widely used.

However, in some cases the geoposition cannot be measured with sufficient accuracy, especially if the geoposition sensor used is located inside a building or vehicle. In this case in particular, a signal from a geoposition sensor may be subject to errors, for example because a signal required by the geoposition sensor is shielded or screened. The installation location can be relatively hidden, making it difficult to locate the sensor despite a relatively accurately measured geoposition. In addition, a geoposition sensor is often not able to measure the height of a location above sea level or above the ground, or not accurately enough. However, this height is often particularly important for locating a sensor, in particular if the sensor is used in a multi-floor (multi-story) building or vehicle, and especially important if several sensors of the same type are used in this building or vehicle. In order to guide the service technician to the installation location using a geoposition sensor, it is also necessary to measure the geoposition of the installation location during the installation phase, save it and make it available.

The invention provides a way for the service technician to locate the installation location in addition to or instead of the geoposition measured by a geoposition sensor during the installation phase, namely with the aid of the reference images. The invention therefore avoids in many cases the disadvantages that would arise if the service technician were to be guided to the installation location solely by means of a signal from a geoposition sensor.

According to the invention, information about the installation location of the sensor is determined for each sensor in the search phase. For this purpose, the or each reference image of this sensor is determined, whereby the or each determined reference image belongs to the data set for this sensor. The search device outputs the determined installation location information in a form that can be perceived by a human.

In one embodiment, the search device provides a service technician with information as to which sensor or sensors the service technician should locate in a search phase, and preferably information as to which respective work is to be performed on the sensor or sensors. The search device is able to capture the following: an identifier of the sensor to be located and/or an input from the service technician that the sensor to be located has now been located and/or an input from the service technician that the required work has been carried out on the sensor. The identifier of the sensor to be located comes, for example, from a work plan (work schedule) for the service technician.

Preferably, the search device captures an input that the sensor to be located has now been located. For example, the search device captures a corresponding confirmation (user input) from the service technician. It is also possible that the search device comprises a reader, whereby the reader is able to read out a unique identifier of the sensor. The unique identifier is applied to a housing of the sensor in the form of a barcode, for example, or stored in an NFC tag, in particular an RFID tag. The unique identifier distinguishes this sensor from at least all other sensors in the sensor arrangement. In the first embodiment, the reader is able to read a barcode; in the second embodiment, it is able to read an NFC tag. If this reader has read the unique identifier of the sensor to be located, the sensor has been located. It is also possible that an alphanumeric character string and/or a symbol is applied to the sensor as a unique identifier in such a way that a human and/or a reader can read this identifier or recognize this symbol.

In one embodiment, the determined installation location information comprises the or at least one, preferably each, reference image of the sensor, wherein this reference image was generated during the installation phase and is part of the data set. The or each determined reference image is transmitted from the central database to the search device. The search device outputs the or at least one, preferably each, transmitted reference image as part of the output installation location information.

This embodiment is relatively easy to implement. The output reference images make it easier for a service technician to locate the installation location. In one implementation, the data sets of the sensors of the sensor arrangement are installed on the search device. In another embodiment, the data set for a sensor to be located—or at least part of the data set—is transmitted from the central computer to the search device as required. The embodiment with the reference images and the embodiment with the geopositions can be combined with each other.

In a preferred embodiment, the operating arrangement comprises a signal-processing image comparison unit. According to this preferred embodiment, not only the installation device but also the search device comprises an image recording device. According to this embodiment, the search phase for the or each sensor to be located comprises the following additional steps, respectively:
The image recording device of the search device generates at least one search image of an environment of the search device, preferably several search images of the environment. Preferably, the service technician uses the image recording device of the search device to generate the search images.
On the one hand, the or at least one, preferably each, reference image is transmitted to the image comparison unit. The or each transmitted reference image was taken during the installation phase, namely from or at the installation location of the sensor, and is part of the data set for the sensor.
On the other hand, the or at least one, preferably every search image that was captured during the search phase is transmitted to the image comparison unit.
The image comparison unit determines the installation location of the sensor in the transmitted search images. For this purpose, the image comparison unit performs a computational image comparison in which at least one search image is compared with at least one reference image of the sensor to be located. Preferably, the image comparison unit searches for an image of the sensor to be located in the transmitted search images, whereby this sensor is shown in at least one reference image. If present, the image comparison unit compares alphanumeric identifiers or symbols or barcodes in the reference images with those in the search images.
The result of the image comparison is transmitted to the search unit. It is also possible that the image comparison unit determines that no search image shows the installation location with sufficient certainty.
The search device determines information about the installation location. To do this, the search device uses the transmitted results of the image comparison.
The search device outputs the determined information about the installation location in at least one form that a human can perceive, in particular visually.

The last two steps are of course only carried out if the image comparison unit has determined the installation location in at least one search image.

Note: The terms “reference image” and “search image” refer to the phase in which the respective image was taken. Technically, the two images can match and, in particular, can have the same data format. They may also have been created using the same image recording device.

The image comparison unit can be a component of the search device or be physically distant (remote) from the search device.

In one implementation, the image comparison unit is a component of the search device and is preferably implemented as a software program. The search images are therefore transmitted to the image comparison unit within the search device and do not need to be transmitted wirelessly. In another implementation, the image comparison unit is spatially remote from the search device. In particular, it is installed on the central computer or on another remote computer. This implementation eliminates the need to provide the search device with a sufficiently large computing capacity and storage capacity. At least temporarily, a bidirectional data connection is established between the image comparison unit and the search device.

In some cases, the installed sensor is at least temporarily and completely or at least partially concealed (hidden) behind an object, from any viewing direction or at least from some viewing directions. For example, the sensor is located in an enclosed space or under a projecting part of a building. The enclosed space is accessible through a door, for example. In order to guide the service technician quickly to the sensor in this situation, preferably a first and a second reference image are generated during the installation phase. The first reference image shows the sensor (the sensor at the sensor installation location). The second reference image shows an object that is located between the image recording device of the installation device and the sensor, for example a closed door to the enclosed space or a container in which the sensor is located. It is possible that several first reference images and/or several second reference images are generated.

An embodiment was described above in which the search device outputs the or each reference image as part of the installation location information. This embodiment can be combined with the embodiment just described, in which at least two reference images are generated, with the first reference image showing the sensor and the second reference image showing the object in front of the sensor. The search device then outputs these two reference images.

The optional image comparison unit compares the search image or at least one search image with both reference images. In the case just described, the search image can show the closed or the open door, whereby the sensor is often visible in the search image when the door is open and not when it is closed.

According to the invention, the installation location information is determined using at least one reference image of a sensor to be located. In one embodiment just described, the image comparison unit compares search images with reference images. In another embodiment, the search unit outputs at least one reference image. Both embodiments require that the or at least some reference images are suitable. More specifically, these reference images must be suitable for the comparison of the search images with the reference images to provide information about the installation location, this information being suitable for the service technician to locate the installation location. As a rule, this presupposes that at least one reference image shows the sensor to be located and no other sensor, at least no other sensor of the same type or appearance, is shown. If reference images are output as part of the installation location information, at least one reference image should show the sensor to be located and ideally no other sensor.

For this reason, in one embodiment, the operating arrangement comprises a signal-processing image evaluation unit in addition to or instead of the image comparison unit. This image evaluation unit is used in the installation phase. The image evaluation unit can be a component of the installation device or spatially remote from the installation device, in particular a component of the central computer or another computer. The or each reference image is transmitted to the image evaluation unit. The image evaluation unit and the image comparison unit can be implemented using different software programs on the same device.

The configuration with the image evaluation unit comprises the following steps:
The image evaluation unit searches each reference image from the sensor's installation location for an image of a sensor in the sensor arrangement. The image evaluation unit does not necessarily determine which sensor is shown in the reference image. Preferably, it distinguishes an image of a sensor of the sensor arrangement from images of other objects and counts how many sensors are shown in the reference image.
In one embodiment, a computer-evaluable catalog with images of sensors is provided. The image evaluation unit uses this catalog to search for images of sensors in the reference images. In one implementation the image evaluation unit uses given contours of the sensors of the sensor arrangement.
The following result is an indication that no suitable reference image is available: Each reference image for a sensor shows either no image of a sensor at all or two images of two sensors that are spatially separated from each other, in particular two sensors that are the same or look the same. If the image evaluation unit has determined this result, it generates a corresponding message.
The installation device outputs this message in at least one form that can be perceived by a human. Preferably, the installation device also outputs each reference image.

This embodiment eliminates the need for the installation technician to check whether at least one suitable reference image is available. It is possible for the installation technician to be informed at the installation location that no suitable reference image is yet available. In response to this, the installation technician can create at least one further reference image already at the installation location. For example, the installation technician takes further reference images at a shorter distance from the sensor and/or from a different viewing direction.

The invention eliminates the need to use a geoposition sensor to guide the service technician to an installation location. In one embodiment, however, both the installation device and the search device each comprise a geoposition sensor. Each geoposition sensor is able to measure its respective own current geoposition. According to this embodiment, the following additional steps are carried out in the installation phase for at least one, preferably for each, sensor of the sensor arrangement:
The geoposition sensor of the installation device measures the geoposition of the installation location where the sensor is or will be installed. More specifically, the geoposition sensor measures its own geoposition, and this often coincides sufficiently accurately with the geoposition of the sensor of the sensor arrangement. Alternatively, the sensor itself comprises a geoposition sensor, and this geoposition sensor measures the geoposition of the installation location.
The measured geoposition of the installation location is transmitted from the installation device to the central computer, preferably together with the reference images and/or with a unique identifier of the sensor and/or with a time stamp. The time stamp indicates the time at which the sensor was installed at the installation location.
The central computer does the following: The data set that is created or supplemented for the sensor also includes the transmitted geoposition of the installation location-more precisely: an identifier of this geoposition. If the data set has already been created, it is supplemented by the transmitted geoposition.

In the search phase, the following step is performed for the or each sensor to be located:
The geoposition sensor of the search device measures its own geoposition, i.e. the geoposition of the search device, at least once. Preferably, the geoposition sensor of the search device measures its own geoposition several times during the search phase, and the search device determines how the distance between the current measured geoposition and the geoposition of the installation location changes.

It is possible to combine the embodiment in which the search device comprises a geoposition sensor with the embodiment in which an image recording device of the search device generates search images and an image comparison unit compares the search images with the reference images. In this combination, the respective measurement of the geoposition is preferably carried out while the image recording device of the search device generates the or one search image.

According to the invention, information about the installation location is determined and the search device outputs this installation location information. The reference images are used for the determination, in one embodiment the result of an image comparison between the search images and the reference images. According to the embodiment just described, the search device additionally uses the following information as components of the installation location information:
the stored geoposition of the installation location of a sensor to be located, this geoposition being measured during the installation phase, transmitted to the central computer, and stored as part of the data set for the sensor, and
the or each geoposition measured by the geoposition sensor of the search device during the search phase.

According to a preferred embodiment, a first sub-phase and a subsequent second sub-phase are carried out in the search phase. In the first sub-phase, a signal from a geoposition sensor is used to guide a service technician to the installation location of a sensor to be located—or at least in the vicinity of the installation location. The limitations described above, which occur when using a geoposition sensor, can also become effective in the first sub-phase.

The embodiment with the two sub-phases is preferably combined with the following embodiment: The search device is capable of capturing an identifier of a located sensor and/or an input from a service technician that a located sensor has been located.

In the first sub-phase, preferably geopositions but no reference images and no search images are used. It is also possible that in the first sub-phase the or at least one, preferably each, reference image is transmitted from the installation location of the sensor to be located to the search device. In the search phase, the search device outputs the or each received reference image visually. In this alternative embodiment, neither the installation device nor the search device necessarily comprise a geoposition sensor.

The optional image comparison unit of the search device is not used in the first sub-phase, but is used in the second sub-phase.

The second sub-phase is carried out if the service technician has not yet located the sensor in the first sub-phase-more precisely: if the search device has not captured either the identifier described above or the input described above. In the second sub-phase, at least one reference image is compared with at least one search image in order to guide the service technician from the location at which the service technician is located at the end of the first sub-phase to the installation location. This comparison is preferably carried out by the optional image comparison unit. The geoposition sensor is not necessarily used in the second sub-phase. However, it is possible to use a signal from a geoposition sensor in the second phase as well, in addition to the or each reference image and the search images.

In this embodiment, the signal from the geoposition sensor and optionally the reference images are used to guide the service technician in the first sub-phase to the vicinity of the installation location. At least if the service technician does not locate the installation location in the first sub-phase, additionally the image comparison unit automatically compares the search images and the or at least one reference image with each other in order to guide the service technician to the installation location. In many cases, this combination increases the reliability that the service technician can quickly locate the installation location compared to an embodiment in which only a signal from a geoposition sensor or only reference images are used. In many cases, this combination also reduces the number of search images that need to be taken and be compared with the reference image(s) until the service technician has located the installation location, compared to an embodiment where only reference images and search images are used. This configuration therefore saves bandwidth because fewer images need to be transmitted.

In the embodiment just described with the first sub-phase and the second sub-phase, the second sub-phase is carried out when the first sub-phase has been carried out and the search device has not captured any input that the sensor has now been located, but no corresponding input at all or an explicit input that the search should be continued.

According to the embodiment described above, the result of the image comparison is transmitted to the search device. The search device outputs information about the installation location in at least one form that can be perceived by a human. Different embodiments are possible as to how the search device outputs the installation location information. At least two of these embodiments can be combined with each other.

In one embodiment, the optional image comparison unit searches for a picture of the sensor to be located in the search images. For this search, the image comparison unit uses at least one reference image of the installation location of the sensor to be located. Preferably, the image comparison unit uses a reference image for the search which shows exactly one sensor. In one implementation, a unique identifier or other visually capturable (detectable) feature of the sensor is visible in this reference image.

If the image comparison unit has located the sensor to be located in a search image, the search device performs the following step: The search device outputs the search image, preferably visually, whereby this search image shows the sensor. In this search image, an automatically or manually generated marker preferably shows an image area of the search image, whereby the image area shows a picture of the sensor to be located. Particularly preferably, the image comparison unit has previously generated a marking (label) of this image area, and this marking has also been transmitted to the search device. It is also possible that the search device itself generates this marking. The configuration with the search image and the marking makes it easier to find the sensor at the installation location.

According to this embodiment, a search image is output which shows the sensor to be located. In many cases, this configuration makes it possible to find the sensor even if the sensor's surroundings have changed since the installation phase. This desired effect is because the search image was created in the search phase, not in the previous installation phase, and is therefore up-to-date.

In one embodiment, several search images are taken during the search phase when searching for a sensor. Each search image is compared with the or at least one, preferably with each reference image. For each search image, a match indicator is calculated with the reference image used for comparison. This match indicator is an indicator of how well the search image matches the reference image. The comparisons and calculations are carried out by the image comparison unit. In one embodiment, the image comparison unit only uses image areas that show one sensor each for image comparison. Preferably, the image comparison unit only uses images that show one sensor in each case, i.e. in particular no reference image that shows an object between the sensor and the camera. If several reference images are stored in the data set for a sensor, it is preferred that several match indicators are calculated for each search image. The largest of these match indicators is preferably used as the overall matching indicator of the search image.

At least one search image is selected. The or each selected search image is output as part of the installation location information on the search device. For example, the search image with the largest match indicator is selected. Or each search image is selected whose match indicator is greater than a predetermined lower matching threshold. In one embodiment, the image comparison unit selects the search images. An identifier of the selected search images is transmitted to the search device. It is also possible that the match indicators are transmitted to the search device and that the search device itself selects search images, depending on the match indicators.

It is possible that no search image is selected, for example because no search image has a sufficiently large match indicator. In this case, the search device preferably issues a message to the service technician, whereby the message includes a request to create at least one further search image.

In many cases, the or each search image output makes it easier for a service technician to locate the sensor. Thanks to the match indicators, there is less risk of an unsuitable or incorrect search image being output.

According to the invention, the search device outputs installation location information. In one embodiment, this installation location information comprises an identifier of a path (way/route) that leads to the sensor to be located. This path preferably begins at a location at which at least one search image has been generated or to which the service technician has been guided on the basis of a signal from the geoposition sensor of the search device and/or on the basis of an output reference image, in particular in the first sub-phase mentioned above. The search device determines this path. According to this embodiment, when generating a search image, the search device measures the viewing direction in which this search image has been created. This viewing direction refers to a global coordinate system, for example. To determine the path to the installation location, the search device uses
the result of the image comparison and
the determined viewing directions of the search images, optionally additionally
the geoposition of the installation location measured during the installation phase, and
the respective geoposition at which a search image was generated, i.e. which was measured during the search phase.

The embodiment that the search device determines and outputs a path to the installation location makes it easier to locate the installation location in many cases, even if this installation location is relatively hidden.

In one embodiment, at least one sensor of the sensor arrangement is installed in a building or vehicle with several floors (stories). In order for a service technician to locate this sensor, he/she must locate the sensor on the correct floor. In one embodiment, at least one reference image generated by this sensor during the installation phase shows an identifier of the floor on which the sensor is installed. This reference image is also part of the data set for the sensor. This reference image does not necessarily show, but may show, the sensor itself. During the search phase, this reference image makes it easier for the service technician to find the correct floor.

In one embodiment, at least one sensor of the sensor arrangement comprises a communication unit. The search device also comprises a communication unit. Thanks to the two communication units, the sensor is able to transmit a signal to the search device. For example, the search device sends out a request which includes an identifier of a sensor to be located, and the sensor to be located sends a response. It is also possible for the sensor to send regularly messages, for example to inform a remote receiver that the sensor is still intact and active.

In one embodiment, the search phase for at least one sensor to be located comprises the following additional steps:

The current distance between the sensor to be located and the search device is measured at least once. Preferably, this distance is measured repeatedly. In one embodiment, the data connection between the sensor and the search device just described is used to measure the distance. Of course, the distance can only be measured using the data connection if a data connection is actually established. It is possible for the distance to be determined additionally or alternatively using the geoposition of the sensor and the search device described above. If the distance is determined using both the data connection and the geoposition, a plausibility check is possible and redundancy is also created.

The search device outputs at least one of the following pieces of information as part of the installation location information:
an indication for the measured distance,
an indication of whether the measured distance increases, decreases or remains the same,
optionally at least one reference image of the sensor to be located,
optionally the geoposition of the installation location, and
optionally an identifier of the sensor to be located, whereby this identifier has been transmitted from the sensor to be located to the search device.

In many cases, this embodiment makes it even easier for the service technician to locate the sensor. The service technician is informed of how far away he/she is from the sensor and whether he/she is moving in the right or a wrong direction relative to the sensor.

According to the invention, the or each sensor of the sensor arrangement is capable of measuring a physical quantity. Different configurations are possible as to which physical quantity this is in each case.

In one embodiment, the or each or at least one sensor of the sensor arrangement is a gas measuring device. In a first alternative, the sensor is able to measure the current concentration of a target gas as a physical variable, in particular that of a flammable or combustible or otherwise harmful target gas or also a target gas that is vital for humans, in particular oxygen. In a second alternative, the sensor is able to automatically decide whether the concentration of the target gas is lower or higher than a predefined threshold. Particularly in the case of flammable (combustible) or other harmful target gases, the concentration must not be greater than a predetermined upper threshold. Conversely, the concentration of oxygen must be greater than a predetermined lower threshold. The target gas can also be an anesthetic agent in a gas mixture or another component of this gas mixture that is delivered to a patient, or anesthetic agents in the ambient air. The anesthetic concentration in the gas mixture should lie within a predetermined range, which is generally limited both upwards and downwards by a threshold.

It is also possible that the or at least one sensor of the sensor arrangement is able to measure an ambient condition, in particular the ambient temperature, the ambient humidity or the ambient pressure or the wind direction and/or wind strength and/or light intensity. The sensor or a sensor can also be configured to measure the amount of precipitation or to detect fire or smoke or dust or dew or fog. Another application is that the sensor measures a sound level or a sound frequency or the intensity or wavelength range of electromagnetic radiation. For example, the sensor is able to use ultrasound to detect a leak in a pipe or other fluid guidance unit or to rule out (exclude) the possibility of a leak. Or the sensor can measure a level of noise or radiation exposure at a particular location. The sensor may also include a motion detector or noise detector, where the motion/noise detector is capable of detecting motion or noise in a sensing area.

A sensor within the meaning of the claims can also be a camera that generates images in the visible range or also in the infrared range or ultraviolet range. A sensor described above may comprise such a camera.

One possible application is as follows: The sensor arrangement comprises a camera and a gas measuring device and is used to monitor a work site where at least one worker is performing a task. The signals from these two sensors are transmitted to a remote control center. This enables an operator at the control center to detect at least one of the following undesirable events in particular: A dangerous target gas is leaking at the work site. An accident has occurred at the work site. A person at the work site does not wear the prescribed protection equipment.

In a preferred embodiment, at least one sensor, preferably each sensor, of the sensor arrangement comprises a communication unit. The or each communication unit is capable of generating a message and causing the message to be transmitted to a spatially remote receiver. This message comprises information about at least one measurement result of the or one sensor of the sensor arrangement and preferably also a time stamp of the measurement. The measurement result comprises a measured value of a physical variable and/or a statement as to whether the current value of the variable falls within a specified value range or not. This message is transmitted to the remote receiver by radio waves and/or by cable. Preferably, the receiver comprises an output unit on which a signal received from a sensor is output in at least one form that can be perceived by a human. Preferably, the communication unit is capable of repeatedly generating and transmitting such a message.

Preferably, each sensor of the sensor arrangement comprises its own power supply unit, but not necessarily an output unit, whereby this output unit outputs a message about a measured target gas concentration in a manner that can be perceived by a human. In general the sensor comprises an output unit which outputs a sensor state. It is also possible that at least one sensor can be permanently or at least temporarily connected to a stationary power supply network.

The invention is described below by means of embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing steps in the installation phase: three gas measuring devices (sensors) are installed and at least one reference image is taken of each sensor installation location;

FIG. 2 is a schematic detail view showing the configuration according to FIG. 1 from the top and showing two reference images of the same installation location that are generated;

FIG. 3 is a schematic view showing a further step in the installation phase: a data set is created in the central database for each of the three gas measuring devices, which includes at least one reference image;

FIG. 4 is a schematic view showing a configuration of the search phase: the reference images of the gas measuring device found are output;

FIG. 5 is a schematic view showing a first sub-phase of the search phase: the geoposition of the gas measuring device to be located is determined and used;

FIG. 6 is a schematic view showing a step of a second sub-phase of the search phase:

the gas measuring device to be located is recognized in a search image;

FIG. 7 is a schematic view showing a step the second sub-phase of the search phase: an object behind which the gas measuring device is located is detected;

FIG. 8 is a schematic view showing a step of the second sub-phase of the search phase: a path to the installation location is calculated based on the viewing directions when generating the search images;

FIG. 9 is a schematic view showing a step of the second sub-phase of the search phase: the path calculated according to FIG. 8 is displayed in two ways;

FIG. 10 is a schematic view showing a step of the second sub-phase of the search phase: the reference image and the measured distance are displayed on the screen of the search device.

### DESCRIPTION OF PREFERRED EMBODIMENTS

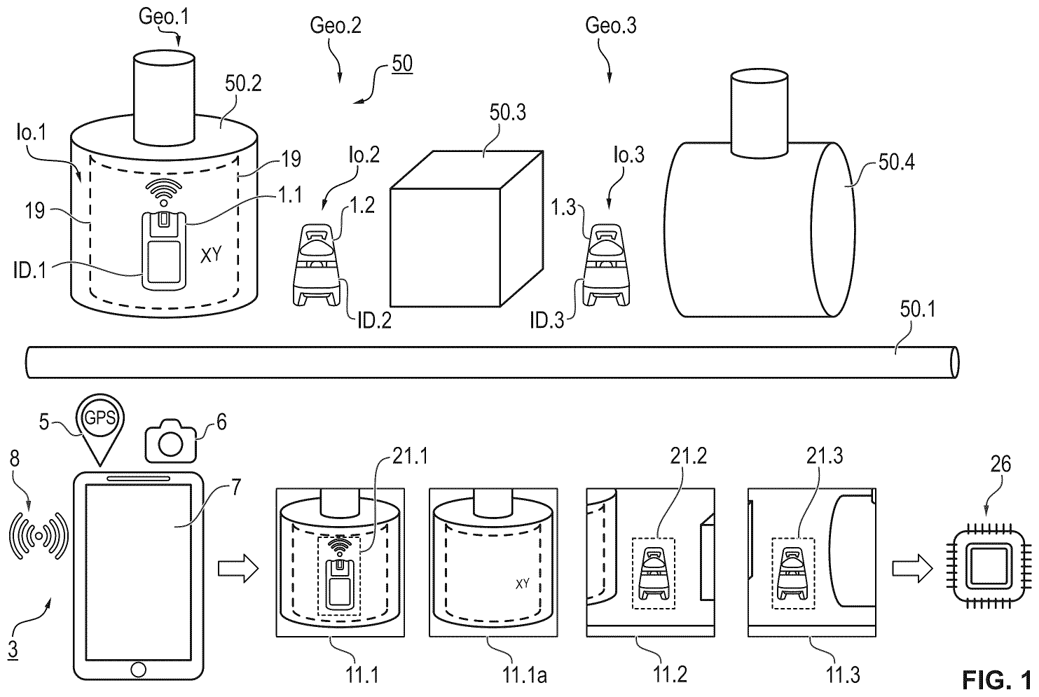

Referring to the drawings, in the embodiment examples, the invention is used in a refinery or other production plant 50. This production plant 50 comprises several components 50.1, 50.2, 50.3, 50.4, which are shown schematically in FIG. 1. In the embodiment example, the sensors of the sensor arrangement are gas measuring devices.

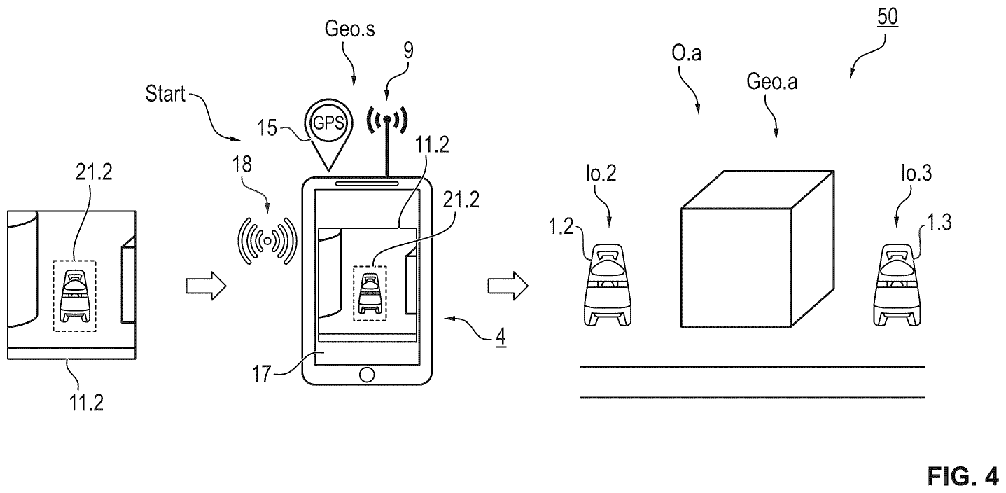

The invention comprises an installation phase and a subsequent search phase. FIG. 1 to FIG. 3 refer to the installation phase, FIG. 4 to FIG. 10 to the search phase. The figures are not necessarily to scale.

In the installation phase, an installation technician installs several stationary gas measuring devices of a gas measuring arrangement at different locations in the production plant 50. By way of example, three gas measuring devices 1.1, 1.2, 1.3 are shown in FIG. 1, which are installed at three different and spatially separated installation locations Io.1, Io.2, Io.3. The process of installing a gas measuring device 1.1, 1.2, 1.3 may include the step of fixing the gas measuring device 1.1, 1.2, 1.3 to a wall or a ceiling and optionally connecting the gas measuring device 1.1, 1.2, 1.3 to a stationary power supply network and/or a data connection network. It is also possible that the gas measuring device 1.1, 1.2, 1.3 is placed on a floor or other surface. Typically, the process also includes at least one of the steps of checking, calibrating and commissioning the gas measuring device 1.1, 1.2, 1.3 at the installation location Io.1, Io.2, Io.3.

Each gas measuring device 1.1, 1.2, 1.3 of the embodiment example is configured to detect at least one target gas in its environment and/or to measure the concentration of the target gas. The or each target gas to be detected is harmful to humans if it occurs in excessive concentrations, and is in particular a flammable and/or toxic gas.

The gas measuring devices 1.1, 1.2, 1.3 each remain at a specific location in the production plant 50 during use. It is possible that a gas measuring device 1.1, 1.2, 1.3 is connected to a stationary power supply network. It is also possible that a gas measuring device 1.1, 1.2, 1.3 comprises its own power supply unit.

In the example shown, each gas measuring device 1.1, 1.2, 1.3 is able to transmit an alarm and optionally a measured target gas concentration to a remote control center (not shown) if the measured target gas concentration is too high, and also preferably regularly a message that the gas measuring device 1.1, 1.2, 1.3 is still functional. The gas measuring device 1.1 is connected to this control center wirelessly via radio waves, while the other two gas measuring devices 1.2, 1.3 are connected to the control center via a wired data connection (not shown).

These implementations are only examples. In the control center, messages from the gas measuring devices are output in at least one form that can be perceived by a human.

In the embodiment example, each gas measuring device 1.1, 1.2, 1.3 comprises a unique identifier ID.1, ID.2, ID.3. In one embodiment, this unique identifier is applied to a surface of the gas measuring device 1.1, 1.2, 1.3 in a machine-readable form, for example as a sequence of alphanumeric characters, a barcode or an RFID chip. In another implementation, the identifier ID.1, ID.2, ID.3 is applied as an alphanumeric character string in such a way that a human can read the identifier.

The installation technician carries a mobile data-processing device 3, which acts as the installation device within the meaning of the claims. By way of example, a smartphone 3 is shown in FIG. 1. In the embodiment example, the installation device 3 comprises a camera or other image recording device 6, a geoposition sensor 5, a reader (scanner) 8, an output unit/input unit, for example a touch screen 7, as well as a computing unit (processor) and a data memory. The image recording device 6 is capable of generating digital images of its surroundings. These images are referred to below as reference images. The geoposition sensor 5 is able to measure its own geoposition. The reader 8 is able to read a machine-readable identifier ID.1, ID.2, ID.3 on a gas meter 1.1, 1.2, 1.3.

The installation technician causes the installation device 3 to perform the following steps after the installation technician has installed and commissioned a gas measuring device 1.1, 1.2, 1.3 at a specific location in the production plant 50:
The reader 8 reads the identifier ID.1, ID.2, ID.3 of the gas measuring device 1.1, 1.2, 1.3. Preferably, the read identifier ID.1, ID.2, ID.3 is output and the installation technician checks whether the identifier ID.1, ID.2, ID.3 has been read correctly or not. Or the installation technician enters the identifier ID.1, ID.2, ID.3 into the installation device 3.
The image recording device 6 generates at least one reference image 11.1, 11.1a, 11.2, 11.3 of the installation location Io.1, Io.2, Io.3 at which the gas measuring device 1.1, 1.2, 1.3 is installed.
At least one reference image 11.1, 11.2, 11.3 shows the gas measuring device 1.1, 1.2, 1.3.
Preferably, at least one further reference image shows an object which in many cases is located at least temporarily between the gas measuring device 1.1, 1.2, 1.3 and a human in the vicinity of the gas measuring device 1.1, 1.2, 1.3. For example, the reference image shows a closed door to a room in which the gas measuring device 1.1, 1.2, 1.3 is located, or another object that visually obscures the gas measuring device 1.1, 1.2, 1.3 from at least one viewing direction.
Preferably, the installation technician or an optional image evaluation unit 26 marks an area 21.1, 21.2, 21.3 in each reference image 11.1, 11.2, 11.3, which shows the gas measuring device 1.1, 1.2, 1.3—provided that the reference image 11.1, 11.2, 11.3 shows the gas measuring device 1.1, 1.2, 1.3.
The geoposition sensor 5 measures at least approximately the respective geoposition Geo.1, Geo.2, Geo.3 of the installation location Io.1, Io.2, Io.3 at which the gas measuring device 1.1, 1.2, 1.3 has been installed. Alternatively, a sensor 1.1, 1.2, 1.3 itself measures the geoposition of its own installation location Io.1, Io.2, Io.3, and the measured geoposition is transmitted to the installation device 3.

Those gas measuring devices for which the steps just described are carried out in the installation phase function together as the sensor arrangement within the meaning of the claims.

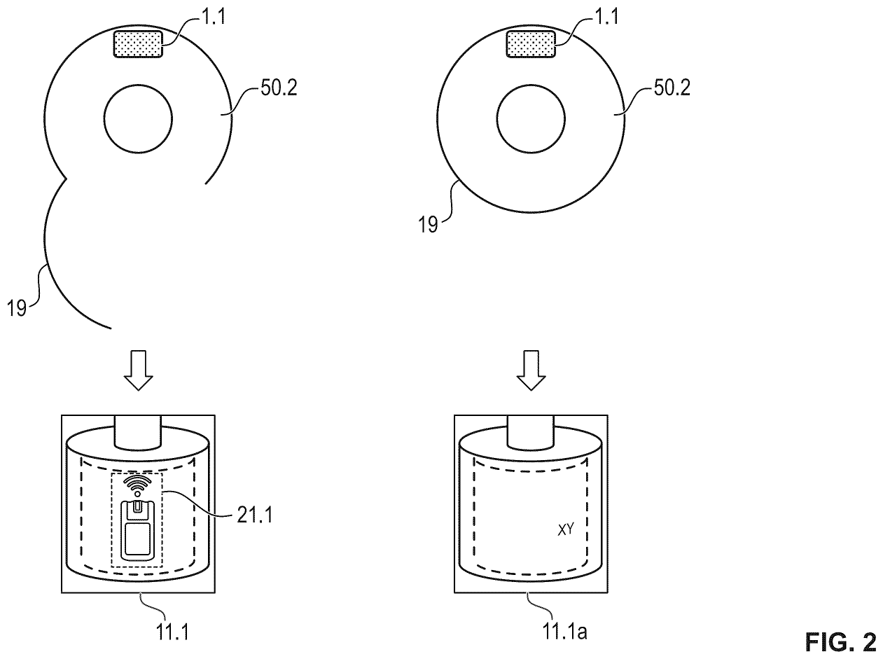

FIG. 1 and FIG. 2 show schematically that a reference image 11.1 is generated by the gas measuring device 1.1 in an enclosed space 50.2 and a further reference image 11.1a is generated from the outside of the enclosed space 50.2. FIG. 1 is a side view, FIG. 2 is a top view. A door 19 can optionally close or open the enclosed space 50.2 and bears a marking XY. FIG. 2 shows on the left the reference image 11.1 with the gas measuring device 1.1 when the door 19 is open. FIG. 2 shows on the right how the reference image 11.1a is produced when the door 19 is closed, whereby the closed door 19 with the marking XY is visible in the reference image 11.1a, but not the gas measuring device 1.1.

In one embodiment, the image evaluation unit 26 automatically searches for a gas measuring device 1.1, 1.2, 1.3 in the reference images 11.1, 11.1a, 11.2, 11.3 of an installation location Io.1, Io.2, Io.3. The image evaluation unit 26 checks whether the following undesirable situation has occurred: In each reference image 11.1, 11.1a, 11.2, 11.3 of an installation location Io.1, Io.2, Io.3, either no image of a gas measuring device 1.1, 1.2, 1.3 is detected at all, or the images of two different and spatially spaced gas measuring devices 1.1, 1.2, 1.3 are detected. The undesired situation has occurred when the image evaluation unit 26 has achieved this result with sufficient certainty. In this case, the image evaluation unit 26 generates a corresponding message. This message is transmitted to the installation device 3. The installation device 3 outputs a corresponding message in at least one form that can be perceived by a human. For example, the installation technician is requested to generate at least one further reference image of the installation location Io.1, Io.2, Io.3, whereby the distance to the gas measuring device 1.1, 1.2, 1.3 is less than the previous reference images of this installation location Io.1, Io.2, Io.3.

Optionally, an application runs on the installation device 3 that supports the installation technician in creating the reference images. This application can be configured in a similar way to instructions that guide a user in creating a panoramic image with a smartphone or other camera. If required, this application supports the installation technician in creating further reference images.

In one embodiment, the application on the installation device enables the installation technician to either mark the gas measuring device shown in the reference image or to enter that this reference image does not show a gas measuring device.

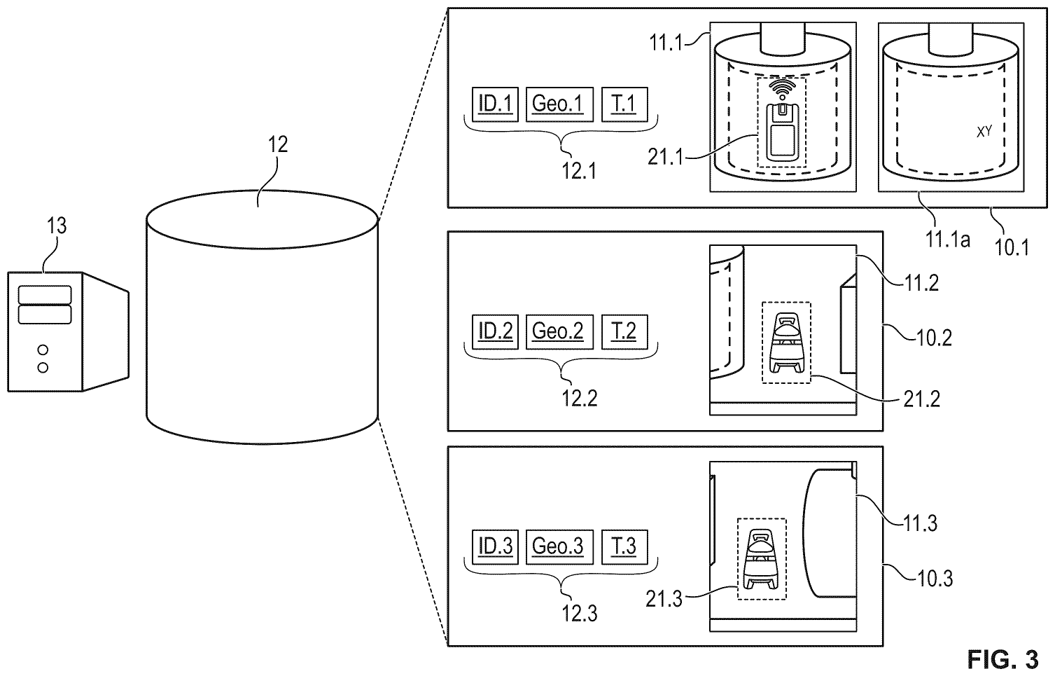

The reference images of the installation locations Io.1, Io.2, Io.3 and the other information about the gas measuring devices 1.1, 1.2, 1.3 are transmitted from the installation device 3 to a remote control center and there to a central computer 13. The central computer 13 has write access and read access to a central database 12, see FIG. 3. A data set is or will be created in the central database 12 for each gas measuring device 1.1, 1.2, 1.3 installed in the production plant 50. The central computer 13 causes a data set 10.1, 10.2, 10.3 to be created in the central database 12 for each installed gas measuring device 1.1, 1.2, 1.3. This data set 10.1, 10.2, 10.3 comprises the following computer-evaluable information about the installed gas measuring device 1.1, 1.2, 1.3:
the unique identifier ID.1, ID.2, ID.3 of the gas measuring device 1.1, 1.2, 1.3,
the geoposition Geo.1, Geo.2, Geo.3 of the installation location Io.1, Io.2, Io.3 at which this gas measuring device 1.1, 1.2, 1.3 is installed,
the time T.1, T.2, T.3 at which the gas measuring device 1.1, 1.2, 1.3 was installed or serviced for the last time,
the or at least one reference image 11.1, 11.2, 11.3, which shows the installed gas measuring device 1.1, 1.2, 1.3 at the installation location Io.1, Io.2, Io.3, wherein preferably in the reference image 11.1, 11.2, 11.3 the area 21.1, 21.2, 21.3 is marked which shows the gas measuring device 1.1, 1.2, 1.3, and
optionally a further reference image 11.1a, which shows an installation location Io.1, but not necessarily a gas measuring device 1.1, 1.2, 1.3.

The identifier ID.1, ID.2, ID.3, the geoposition Geo.1, Geo.2, Geo.3 and the time T.1, T.2, T.3 together form the information 12.1, 12.2, 12.3 in data set 10.1, 10.2, 10.3.

In the application just mentioned, the installation phase comprises the step in which the installation technician installs each gas measuring device 1.1, 1.2, 1.3 at a respective installation location Io.1, Io.2, Io.3, generates the reference images 11.1, 11.1a, 11.2, 11.3 and causes the respective geoposition Geo.1, Geo.2, Geo.3 and preferably the time of installation to be measured. It is also possible that the installation phase is carried out after the gas measuring devices 1.1, 1.2, 1.3 have been installed, for example because the invention is to be applied to an already installed sensor arrangement. The installation technician who carries out the steps of the installation phase therefore does not necessarily install a gas measuring device 1.1, 1.2, 1.3. Preferably, the installation technician who carries the installation device 3 and generates the reference images 11.1, 11.2, 11.3 also carries documentation about the respective installation location Io.1, Io.2, Io.3. Thanks to the invention, these documents are no longer required during the search phase.

From time to time, it is necessary for a service technician to check each gas measuring device 1.1, 1.2, 1.3 installed in the production plant 50. In addition, an event may require the service technician to check a gas measuring device 1.1, 1.2, 1.3 and repair it if necessary. Examples of such events are:
The gas measuring device 1.1, 1.2, 1.3 no longer transmits a message to the control center that it is operational.
The gas measuring device 1.1, 1.2, 1.3 has transmitted an error message to the control center, for example about a low charge status of its own power supply unit or poisoning or a failure of the actual sensor.
The gas measuring device 1.1, 1.2, 1.3 is contaminated with harmful target gases to such an extent that it must be replaced.
The intended service life of the gas measuring device 1.1, 1.2, 1.3 has been reached.

In one implementation, a service technician selects a gas measuring device 1.1, 1.2, 1.3 on the production plant 50 using the unique identifier ID.1, ID.2, ID.3. In another implementation, the service technician is given a work order. This work order specifies the respective identifier ID.1, ID.2, ID.3 of each gas measuring device 1.1, 1.2, 1.3 on which work is now to be performed and specifies the respective work to be performed. The work order is preferably generated by the central computer 13 using the data sets and in particular the time stamps T.1, T.2, T.3. This work must be carried out at least partially on site, i.e. it cannot be carried out exclusively remotely. It is therefore necessary for the service technician to locate the or each gas measuring device on which work is currently to be carried out on the production plant 50. Such a gas measuring device is referred to below as a “gas measuring device to be located”.

In the embodiment example, work is to be carried out on the two gas measuring devices 1.1 and 1.2, and therefore these two gas measuring devices 1.1, 1.2 are to be located in the search phase. FIG. 4 to FIG. 10 illustrate an example of how the service technician is guided to the installation location Io.1 or Io.2 where the gas measuring device 1.1 or 1.2 is installed. The service technician carries a mobile data-processing device, for example also a smartphone 4. This mobile device 4 acts as the search device within the meaning of the claims. It is possible that the same device functions both as the installation device 3 and as the search device 4. The search device 4 comprises
a geoposition sensor 15,
an image recording device 16,
a screen 17 that is preferably touch-sensitive (“touch screen”),
optionally a communication unit with an antenna 9 and
optionally an RFID reader 18.

The information 12.1, 12.2 and each reference image 11.1, 11.1a, 11.2 from the data set 10.1, 10.2 for the gas measuring device 1.1, 1.2 to be located are transmitted to the search device 4. For example, a wireless data connection is established at least temporarily between the central database 12 and the search device 4, for which the communication unit with the antenna 9 is used. In the embodiment example, the transmitted information 12.1, 12.2 comprises the geoposition Geo.1, Geo.2 at which the gas measuring device 1.1, 1.2 is installed and, in one embodiment, the reference images 11.1, 11.1a, 11.2.

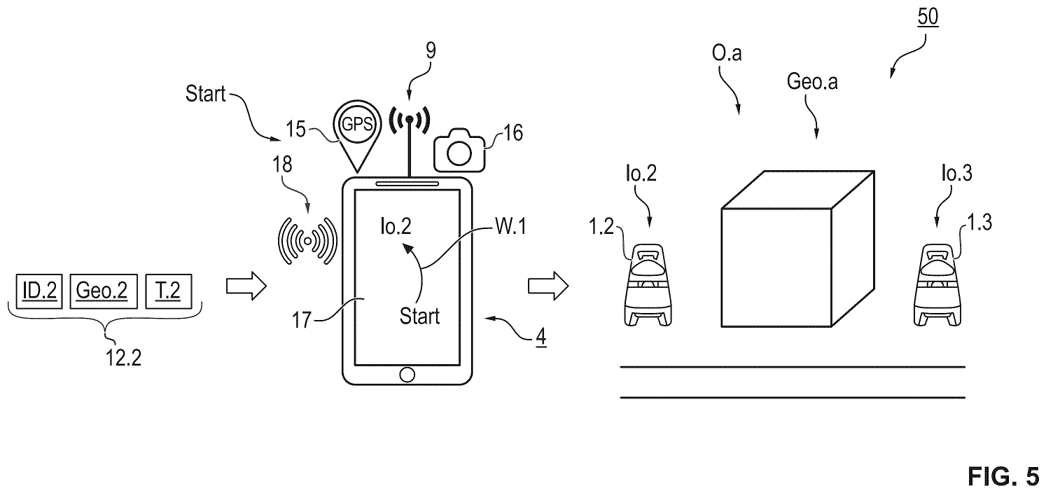

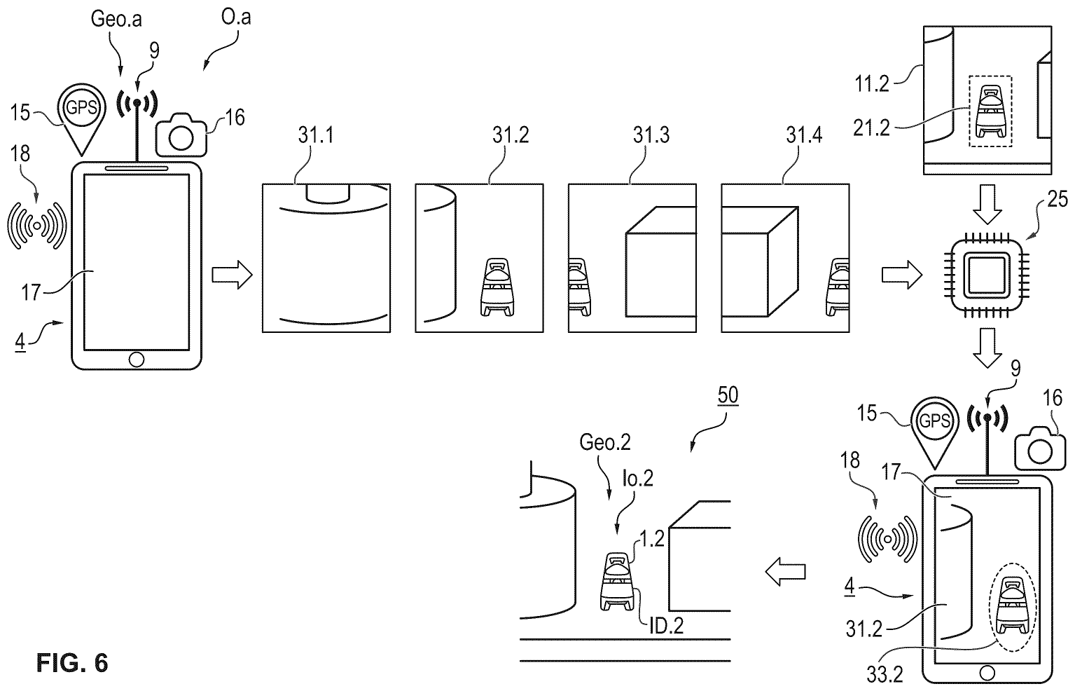

In the embodiment example, the search phase comprises a first sub-phase, which is illustrated in FIG. 4 and FIG. 5, and a second sub-phase, which is illustrated in FIG. 6 to FIG. 10.

In the first sub-phase, the search device 4 guides the service technician from a starting point Start with the geoposition Geo.s to the installation location Io.2, or at least to a location in the vicinity of the installation location Io.2. For this purpose, the search device 4 uses the transmitted geoposition Geo.2 as well as its own current geoposition, which is repeatedly measured with the geoposition sensor 15. The search device 4 calculates a path W.1 from the starting position Start to the installation location Io.2 and displays information about the calculated path W.1 on the screen 17. For this purpose, the search device 4 uses the geoposition Geo.s of the starting point Start and the geoposition Geo.2 of the installation location Io.2. Preferably, the reference image 11.2 from the data set 10.2 is displayed on the screen 17 of the search device 4. The marking 21.1 shows where the gas meter 1.2 is shown in the reference image 11.2.

It is possible that the first sub-phase is already sufficient to guide the service technician to the installation location Io.2. However, it is possible that the steps of the first sub-phase alone are not sufficient for the service technician to locate the gas measuring device 1.2 at the installation location Io.2. Some possible reasons for this are
When installing the gas measuring device 1.2, the geoposition Geo.2 of the installation location Io.2 was not measured accurately enough.
The search device 4 measures its own current geoposition only approximately, i.e. with an error.
The gas measuring device 1.2 is relatively difficult to locate at the installation location Io.2 simply due to its geoposition Geo.2, especially because it is concealed at the installation location or is mounted in a building or vehicle with several floors.

FIG. 4 and FIG. 5 show by way of example that the service technician is not guided to the installation location Io.2, but to a location O.a, which is close to the searched installation location Io.2 and has the geoposition Geo.a. For example, a second sub-phase is carried out in response to a corresponding user input. The second sub-phase guides the service technician from location O.a to installation location Io.2. This second sub-phase is illustrated in FIG. 6 to FIG. 10. The second sub-phase comprises the processes described below.

In the embodiment shown in FIG. 4, the search device 4 does not necessarily comprise an image recording device 16. An image comparison unit 25 is also not required. The reference image 11.2 of the installation location Io.2 is determined in the central database 12 as part of the data set 10.2 and transmitted to the search device 4. The search device 4 displays the transmitted reference image 11 on its screen 17.

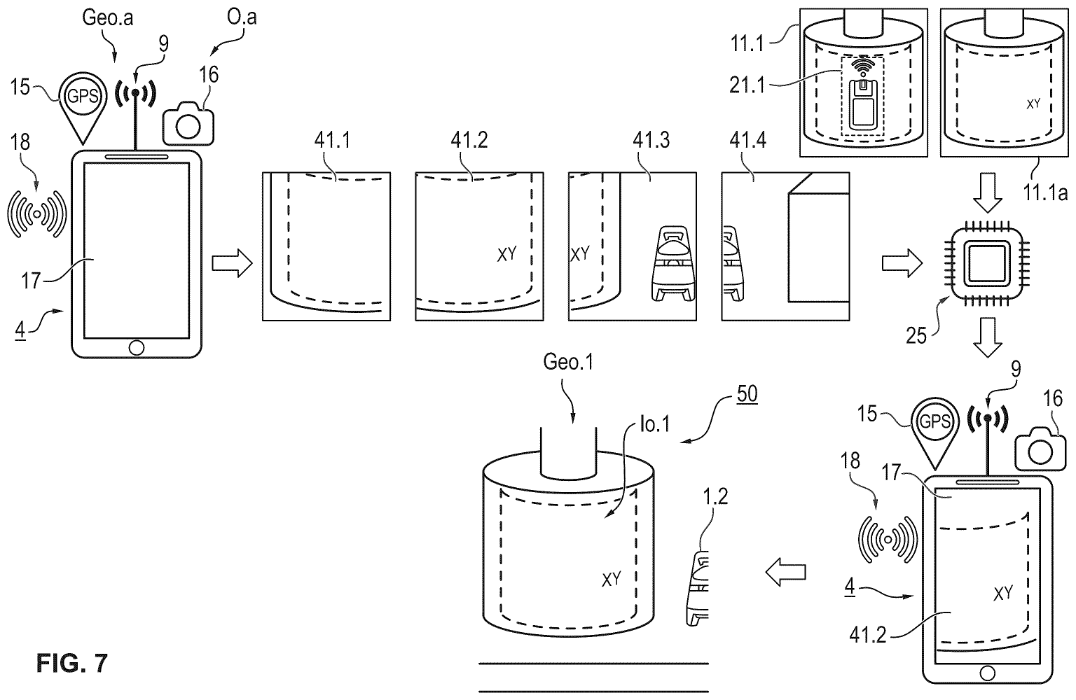

In the embodiment according to FIG. 5 to FIG. 10, an image recording device 16 of the search device 4 and an image comparison unit 25 are used. The service technician causes the image recording device 16 of the search device 4 to take several images of the surroundings of location O.a. These images are referred to as search images. By way of example, four search images 31.1, 31.2, 31.3, 31.4 are shown in FIG. 6 and four search images 41.1, 41.2, 41.3, 41.4 are shown in FIG. 7. For example, the service technician rotates once around his/her own axis at location O.a, and the image recording device 16 generates a sequence of search images.

Optionally, an application runs on the search device 4 that supports the service technician in generating the search images 31.1, 31.2, 31.3, 31.4, 41.1, 41.2, 41.3, 41.4. This application can be configured similarly to an instruction that guides a user to create a panoramic image with the camera of a smartphone or other camera.

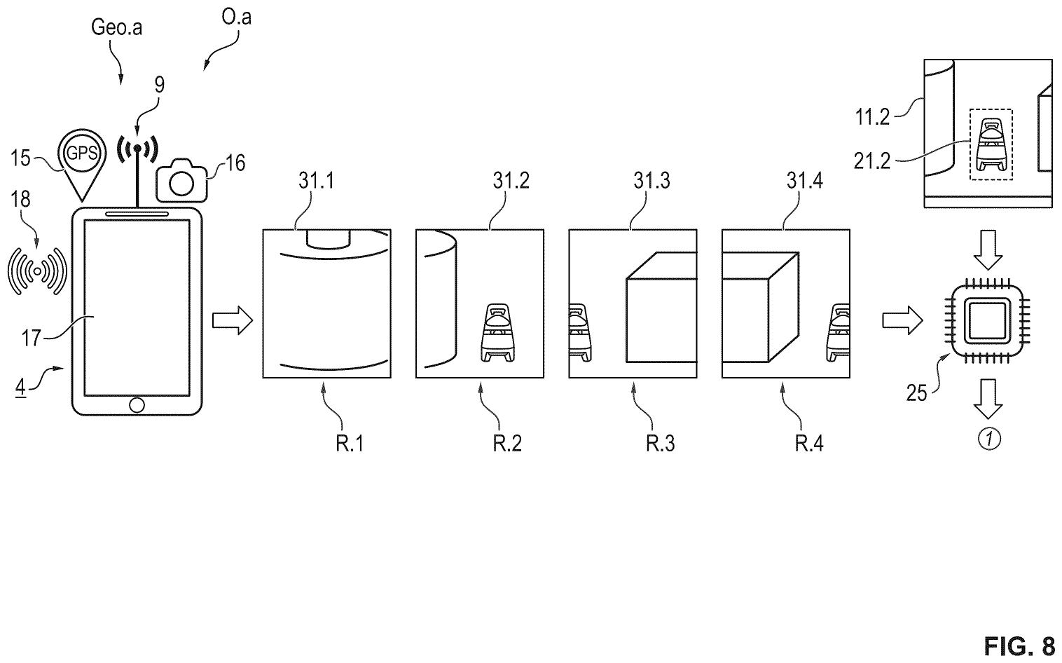

The geoposition sensor 15 measures the geoposition Geo.a of the location O.a at which the search images 31.1, 31.2, 31.3, 31.4 are taken. Preferably, the search device 4 measures the respective viewing direction R.1, R.2, R.3, R.4 in which the image recording device 16 has taken a search image 31.1, 31.2, 31.3, 31.4 of the surroundings, see FIG. 8.

An image comparison unit 25 compares the search images 31.1, 31.2, 31.3, 31.4, 41.1, 41.2, 41.3, 41.4, which the image recording device 16 has produced at location O.a, with the or at least one, preferably each reference image 11.1, 11.1a, 11.2 from the data set 10.1, 10.2.

In one embodiment, the image comparison unit 25 is part of the search device 4. This configuration eliminates the need to transmit data from the search device 4 to another computer. In another embodiment, the image evaluation unit 25 belongs to the central computer 13 or to another spatially remote computer. The search images 31.1, 31.2, 31.3, 31.4, 41.1, 41.2, 41.3, 41.4 are transmitted from the search device 4 to the central computer 13 or to the other computer, and there the image comparison unit 25 compares the search images 31.1, 31.2, 31.3, 31.4, 41.1, 41.2, 41.3, 41.4 with the reference image 11.1, 11.1a, 11.2. The result of the image comparison is transmitted back to the search device 4. The fact that the image comparison unit 25 is arranged at a distance from the search device 4 eliminates the need to provide the search device 4 itself with a sufficiently powerful processor.

In one embodiment, the image comparison unit 25 searches in a reference image 11.1, 11.1a, 11.2 for the image of the gas measuring device 1.1, 1.2 to be located. For this purpose, the image comparison unit 25 preferably uses the marking 21.2. The image comparison unit 25 searches in the search images 31.1, 31.2, 31.3, 31.4, 41.1, 41.2, 41.3, 41.4 for an area showing such a gas measuring device and highlights this area in such a search image.

In the example in FIG. 6, the image of the gas measuring device 1.2 is marked 21.2 in the reference image 11.2. The image comparison unit 25 has recognized an image of the gas measuring device 1.2 to be located in the search image 31.2. The search device 4 displays the search image 31.2 on the screen 17. The image area in which the gas measuring device 1.2 is shown is highlighted by the marking 33.2.

It is possible that the image comparison unit 25 has not found an image of the gas measuring device 1.1, 1.2 in any of the search images 31.1, 31.2, 31.3, 31.4, 41.1, 41.2, 41.3, 41.4. FIG. 7 shows such an example. In this example, the gas measuring device 1.1 is to be located. In the first sub-phase, the service technician was guided to a location O.b with the geoposition Geo.b. The first sub-phase proceeded as described above with reference to FIG. 6. The gas measuring device 1.1 now to be located is located in the enclosed space 50.2, and the door 19 is closed, see FIG. 1 and FIG. 2. At location O.b, the camera 16 generates the search images 41.1, 41.2, 41.3, 41.4. The image comparison unit 25 does not find an image of the gas measuring device 1.1 in any search image 41.1, 41.2, 41.3, 41.4, which is shown in the reference image 11.1 and highlighted by the marking 21.1. The image comparison unit 25, on the other hand, determines an image of a part of the door 19 in the search image 41.2, whereby the image comparison unit 25 detects the reference image 11.1a and in it, for example, the marking XY and/or a particular shape of the door 19. The search device 4 displays the search image 41.2 on the screen 17.

It is also possible that the service technician makes a user input that he/she has not located the gas measuring device 1.2. In one implementation, the service technician is asked to go to another location and take search images again.

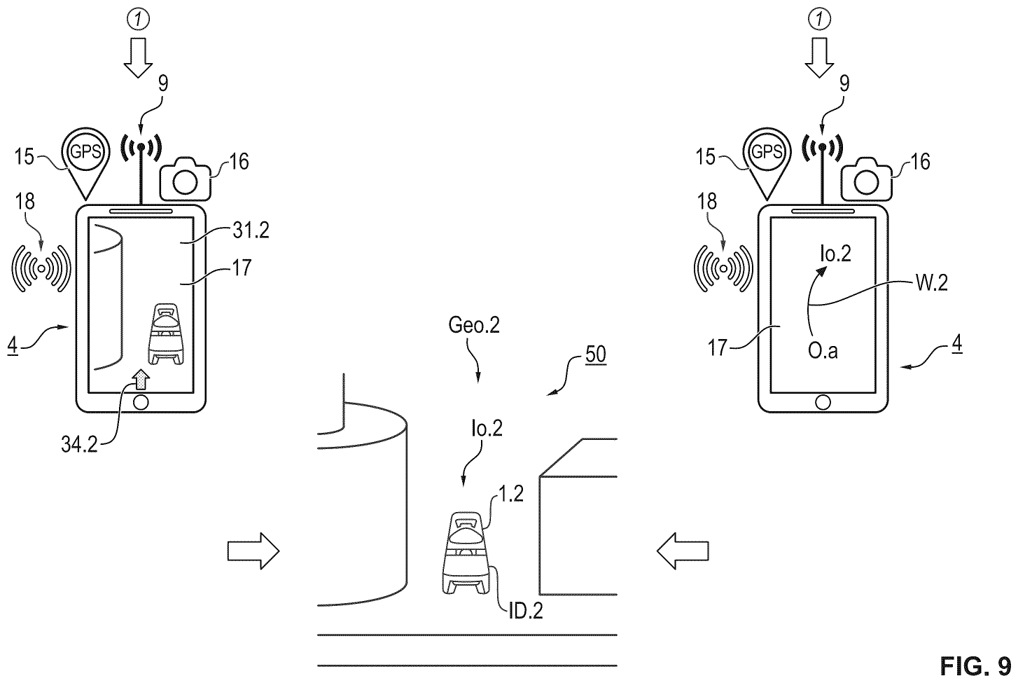

FIG. 8 and FIG. 9 show an alternative or additional implementation. In this example, the gas measuring device 1.2 is again to be located. According to this alternative or additional implementation, the image evaluation unit 25 calculates a path W.2 from the location O.a, where the service technician is currently located, to the installation location Io.2 being searched for—or at least the direction from the location O.a to the installation location Io.2. For this purpose, the image evaluation unit 25 uses
the measured geoposition Geo.a of the location O.a,
the transmitted geoposition Geo.2 of the installation location Io.2,
the reference image 11.2,
the search image 31.1, 31.2, 31.3, 31.4 and
the respective viewing direction R.1, R.2, R.3, R.4, from which a search image 31.1, 31.2, 31.3, 31.4 was generated.

The search device 4 displays the calculated path W.2 from location O.a to the searched installation location Io.2 on screen 17. In the example in FIG. 9 on the left, the search image 31.2 is shown on screen 17. An arrow 34.2 is shown in the search image 31.2, indicating the direction to the installation location Io.2. In the example in FIG. 9 on the right, an arrow is shown that specifies a path W.2 from location O.a to installation location Io.2. This arrow can, for example, be superimposed on a section of a map or a floor plan (not shown) of the production plant 50.

It is possible that the service technician will now locate the gas measuring device 1.1, 1.2 they are looking for. In one implementation, the optional reader 18 reads the unique identifier ID.1, ID.2 of the gas measuring device 1.1, 1.2. This is a confirmation that the service technician has located the correct gas measuring device 1.1, 1.2. In another implementation, the service technician reads an identifier ID.1, ID.2 in the form of an alphanumeric character string on a surface of the gas measuring device 1.1, 1.2 and enters a confirmation that the gas measuring device 1.1, 1.2 being searched for has now been located.

If the service technician has not located the gas measuring device 1.1, 1.2, the second sub-phase just described is preferably carried out again. Before the second sub-phase, the service technician is at a location O.c. The steps described above are carried out again, this time with location O.c instead of location O.a. If necessary, the second sub-phase is repeated until the service technician has located the gas measuring device 1.1, 1.2.

In one embodiment, an indicator of the current distance between a gas measuring device 1.x to be located and the search device 4 is measured at least once, preferably several times. Preferably, an automatic attempt is made to establish a wireless data connection between the gas measuring device 1.x and the search device 4. The wireless connection uses, for example, a protocol according to the Bluetooth or Bluetooth Low Energy (BLE) standard or WLAN. The search device 4 comprises a receiver for a wirelessly transmitted signal and preferably also a transmitter. Accordingly, the gas measuring device 1.x comprises a transmitter and preferably a receiver. However, it is also sufficient that the gas measuring device 1.x comprises only a transmitter and the search device 4 comprises only a receiver.

As soon as this data connection is established, the distance is measured at least once. To measure the distance, for example, the strength of a signal transmitted from gas measuring device 1.x to search device 4 is measured. Or the time it takes for a signal to be transmitted from the gas measuring device 4 to the gas measuring device 1.x and back from the gas measuring device 1.x to the search device 4 is measured. The or a signal which is transmitted from the gas measuring device 1.x to the search device 4 preferably comprises a unique identifier of the gas measuring device 1.x, so that the search device 4 can automatically decide which gas measuring device is in its vicinity.

In a preferred embodiment, the search device 4 outputs at least one portion of the following information, in a form that can be perceived by a human, preferably on the screen 17:
the event that a data connection has been established between the search device 4 and the gas measuring device 1.x to be located,
the event that this data connection is interrupted again,
an indication for the measured distance dist,
an indication of whether the measured distance dist increases, decreases or remains the same.

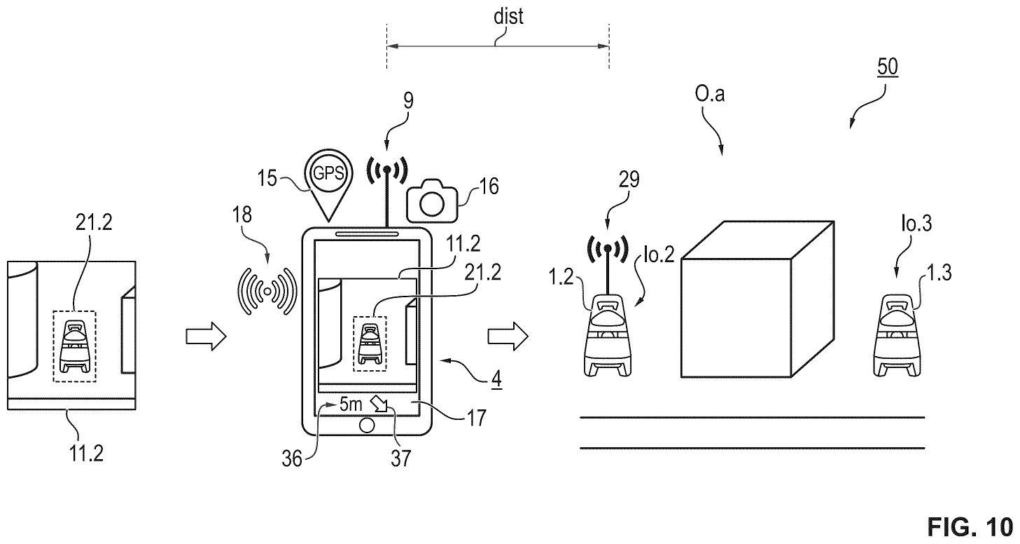

FIG. 10 illustrates this embodiment by way of example. The locating gas measuring device 1.2 comprises a communication unit with an antenna 29. As already mentioned, the locating device 4 comprises a communication unit with an antenna 9. As soon as a wireless data connection is established between the locating device 4 and the gas measuring device 1.2, the distance dist is measured. In the example shown, the search device 4 displays the following information on the screen 17:
the transmitted reference image 11.2 of the gas measuring device 1.2 to be located,
an indication 36 for the measured distance dist (here: 5 m) and
an indication 37 that the distance dist has become smaller, i.e. the service technician is on the right track (path).

In a further development of this embodiment, the search device 4 is additionally able to determine in which direction a path from the search device 4 to the gas measuring device 1.x runs. Preferably, the search device 4 is able to determine the direction from which a signal comes from the gas measuring device 1.x. The search device 4 comprises at least two receivers for a signal from the gas measuring device 1.x. The direction is preferably also displayed on the screen 17.

While specific embodiments of the invention have been shown and described in detail to illustrate the application of the principles of the invention, it will be understood that the invention may be embodied otherwise without departing from such principles.

List of reference characters

| | |
|---|---|
| 1.1, 1.2, 1.3 | Stationary gas measuring devices (sensors), installed at |
| | the installation locations Io.1, Io.2, Io.3, belong to the |
| | sensor arrangement of the embodiment example |
| 3 | Mobile data-processing installation device, such as a |
| | smartphone, comprises the camera 6, the geoposition |
| | sensor 5, the reader 8 and the screen 7 |
| 4 | Mobile data-processing search device, for example a |
| | smartphone, comprises the camera 16, the geoposition |
| | sensor 15, the reader 18 and the screen 17 |
| 5 | Geoposition sensor of the installation device 3 |
| 6 | Camera of the installation device 3, generates the |
| | reference images 11.1, 11.1a, 11.2, 11.3 |
| 7 | Touch-sensitive screen of the installation device 3 |
| 8 | Reader for reading an ID.1, ID.2, ID.3 identifier, |
| | belongs to the installation device 3 |
| 9 | Antenna, belongs to a communication unit of the search |
| | device 4 |
| 10.1, 10.2, | Data sets in the central database 12 for the gas |
| 10.3 | measuring devices 1.1, 1.2, 1.3, each of the data sets |
| | include information on the unique identifier ID.1, ID.2, |
| | ID.2, the installation location Io.1, Io.2, Io.3, the time |
| | T.1, T.2, T.3 of the last maintenance and the reference |
| | image 11.1, 11.1a, 11.2, 11.3 |
| 11.1, 11.1a, | Reference images of the installation locations Io.1, Io.2, |
| 11.2, 11.3 | Io.3, generated by camera 6 |
| 12 | Central database in which a data set 10.1, 10.2, 10.3 is |
| | stored for each gas measuring device 1.1, 1.2, 1.3 on the |
| | production plant 50 |
| 13 | Central computer, has read and write access to the |
| | central database 12, receives messages from the |
| | installation device 3 |
| 15 | Geoposition sensor of the search device 4 |
| 16 | Camera of the search device 4, generates the search |
| | images 31.1, . . . , 31.4, 41.1, . . . , 41.4 |
| 17 | Touch-sensitive screen of the search device 4 |
| 18 | Reader for reading an identifier ID.1, ID.2, ID.3, belongs |
| | to the search device 4 |
| 19 | Door to enclosed room 50.2, marked XY |
| 21.1, 21.2, | Markings indicating the area in which the gas measuring |
| 21.3 | device 1.1, 1.2, 1.3 is shown in the reference images |
| | 11.1, 11.2, 11.3 |
| 25 | Image comparison unit, automatically compares the |
| | search images 31.1, 31.2, 31.3, 31.4 with the reference |
| | image 11.2 |
| 26 | Image evaluation unit, searches in the reference images |
| | 11.1, 11.1a, 11.2, 11.3 for an image of a gas measuring |
| | device |
| 29 | Antenna, belongs to a communication unit of the gas |
| | measuring device 1.2 |
| 31.1, 31.2, | Search images taken by imaging device 16 during the |
| 31.3, 31.4 | search for gas measuring device 1.2 at location O.a |
| 33.2 | Marking of the area in which the search image 31.2 |
| | shows the gas measuring device 1.2 is displayed |
| | together with the search image 31.2 on screen 17 |
| 34.2 | Arrow superimposed on the search image 31.2 to show |
| | the way to the installation location Io.2 |
| 36 | Indication of the measured distance dist between the |
| | search device 4 and the gas measuring device 1.2 to be |
| | located is displayed on the screen 17 |
| 37 | Indication that the distance dist between the search |
| | device 4 and the gas measuring device 1.2 is decreasing |
| | is displayed on the screen 17 |
| 41.1, 41.2, | Search images taken by imaging device 16 during the |
| 41.3, 41.4 | search for gas measuring device 1.1 at location O.a |
| 50 | Production plant in which the gas measuring devices |
| | 1.1, 1.2, 1.3 are installed comprises the components |
| | 50.1, 50.2, 50.3, 50.4 |
| 50.1, 50.2, | Components of the production plant 50 |
| 50.3, 50.4 | |
| dist | Measured distance between the search device 4 and the |
| | gas measuring device 1.2 to be located |
| Geo.1, | Geopositions of the installation locations Io.1, Io.2, Io.3 |
| Geo.2, Geo.3 | |
| Geo.a | Geoposition of the location O.a |
| Geo.b | Geoposition of the location O.b |
| Geo.s | Geoposition of the starting point Start |
| ID.1, ID.2, | Unique identifiers of the gas measuring devices 1.1, 1.2, |
| ID.3 | 1.3, are machine-readable in one embodiment |
| Io.1, Io.2, | Installation locations where the stationary gas |
| Io.3 | measuring devices 1.1, 1.2, 1.3 are installed |
| T.1, T.2, T.3 | Time at which the gas measuring device 1.1, 1.2, 1.3 |
| | was installed or serviced last |
| O.a | Location to which the service technician is guided by |
| | comparing the current geoposition of the search device |
| | 4 with the geoposition Geo.2, at the same time location |
| | at which the search images 31.1, 31.2, 31.3, 31.4 are |
| | made, has the geoposition Geo.a |
| O.b | The location at which the search images 41.1, 41.2, |
| | 41.3, 41.4 are created has the geoposition Geo.b |
| R.1, R.2, R.3, | Viewing directions in which the camera 16 is directed |
| R.4 | when generating the search images 31.1, 31.2, 31.3, |
| | 31.4 |
| Start | The starting point at which the installation technician |
| | begins the search for the installation location Io.2 has |
| | the geoposition Geo.s |
| W.1 | Path from the starting point Start to the installation |
| | location Io.2, calculated by the search device 4 based on |
| | the geopositions Geo.a and Geo.2, is displayed on |
| | screen 17 |
| W.2 | Path from location O.a to installation location Io.2, |
| | calculated by search device 4 on the basis of search |
| | images 31.1, 31.2, 31.3, 31.4 and reference image 11.2, |
| | is displayed on screen 17 |

What is claimed is:

1. An operating process for operating a sensor arrangement which sensor arrangement comprises at least one sensor, the sensor being configured to measure a physical quantity and/or to check whether the physical quantity lies within a predetermined value range or not, the sensor being configured to be installed at a sensor installation location;
providing an operating arrangement, the operating arrangement comprising: a mobile data-processing installation device, which comprises an image recording device; a mobile data-processing search device; a central computer; and a central database;
carrying out an installation phase to install the or every sensor of the sensor arrangement as an installed sensor at the respective sensor installation location, the installation phase comprising for every sensor of the sensor arrangement the steps of:
generating at least one reference image of the sensor installation location;
transmitting each generated reference image from the installation device to the central computer; and
with the central computer, creating a data set for the installed sensor in the central database or supplementing an existing data set, wherein after the creation or supplementation the data set comprises the or each transmitted sensor reference image of the sensor installation location; and
carrying out a search phase for locating the or at least one installed sensor of the sensor arrangement, which installed sensor of the sensor arrangement is an installed sensor to be located, for each sensor to be located the search phase comprising the steps of:
determining each reference image in the data set for the installed sensor to be located;
with the or at least one determined reference image, determining installation location information; and
with the data-processing search device, outputting the determined installation location information in a form that can be perceived by a human.

2. An operating process according to claim 1, wherein the step of generating at least one reference image comprises the step of generating at least two reference images of the installation location during the installation phase of at least one sensor, wherein a first reference image shows the installed sensor and a second reference image shows an object which is located between the image recording device and the installed sensor when the second reference image is generated, wherein the object completely or at least partially obscures the installed sensor.

3. An operating process according to claim 1, wherein the operating arrangement additionally comprises a signal-processing image evaluation unit, and the installation phase additionally comprises for every sensor of the sensor arrangement the steps of:
with the image evaluation unit, searching each reference image from the installation location of the sensor for an image of a sensor;
if the image evaluation unit finds in each reference image from the installation location of the sensor either no image of a sensor or two images of two different sensors, generating a corresponding message with the image evaluation unit; and
with the installation device, outputting the message in a form that can be perceived by a human.

4. An operating process according to claim 1,
wherein the determined installation location information comprises the or each reference image comprised by the data set and determined during the installation phase, and
wherein the steps of outputting the determined installation location information for a sensor to be located comprises the step of transmitting the or each determined reference image for the sensor to the search device and outputting the determined reference image by the search device.

5. An operating process according to claim 1,
wherein the search device comprises a search device image recording device, and
wherein the search phase comprises for the or at least one sensor to be located the additional step that the search device image recording device generates at least one search image of an environment of the search device.

6. An operating process according to claim 5,
wherein both the installation device and the search device each additionally comprise a geoposition sensor,
wherein for each sensor of the sensor arrangement the installation phase comprises the additional steps of:
with the geoposition sensor of the installation device, measuring a geoposition of the installation location;
transmitting the measured geoposition of the installation location from the installation device to the central computer; and
with the central computer, supplementing the data set created or supplemented for the sensor with the transmitted geoposition of the installation location of the sensor; and
wherein the search phase comprises for the or every sensor to be located the additional steps of:
with the geoposition sensor of the search device, measuring a geoposition of the search device at least once while the search image is generated; and
with the search device, for determining the installation location information, additionally using the geoposition of the installation location measured during the installation phase and stored in the data set and using each geoposition measured by the geoposition sensor of the search device when generating the search images.

7. An operating process according to claim 5,
wherein the operating arrangement further comprises a signal-processing image comparison unit,
wherein the search phase comprises for the or at least one sensor to be located the additional steps of:
transmitting each reference image included in the data set for the sensor to be located and each search image of the sensor generated in the search phase to the image comparison unit;
with the image comparison unit, computationally performing an image comparison on the transmitted images to determine in at least one search image the installation location of the sensor or to determine that no search image shows the installation location of the sensor;
transmitting a result of the image comparison to the search device; and
with the search device, determining information about the installation location using the result of the image comparison if the image comparison unit has determined in at least one search image the installation location of the sensor.

8. An operating process according to claim 7, further comprising the step of, with the image comparison unit, searching the at least one search image for a picture of the sensor to be located,
wherein the image comparison unit uses for the search for the picture the at least one reference image from the installation location of the sensor to be located, and if at least one search image with a picture of the sensor is located with the reference image of the sensor to be located, the step that the search device outputs the installation location information comprises the step that the search device outputs the at least one search image, which shows the sensor to be located together with a marking of an image area in which the picture of the sensor to be located is shown in the search image.

9. An operating process according to claim 6,
wherein the search phase for the or at least one installed sensor to be located comprises a first sub-phase and a subsequent second sub-phase,
wherein the steps of determining each reference image in the data set for the sensor to be located, determining installation location information with the determined reference image, and with the search device, outputting the determined installation location information are carried out in the second sub-phase, and
wherein the first sub-phase comprises the steps of:
transmitting the stored geoposition of the installation location of the sensor to be located to the search device;
with the geoposition sensor of the search device, measuring at least once a geoposition while the search image is being generated;
with the search device, determining a path from the measured geoposition of the geoposition sensor of the search device to the transmitted geoposition stored in the data set; and
with the search device, outputting information about the determined path in a form that can be perceived by a human.

10. An operating process according to claim 7,
wherein the search phase for the sensor to be located comprises a first sub-phase and a subsequent second sub-phase,
wherein the steps of determining each reference image in the data set for the installed sensor to be located, determining installation location information with the determined reference image; and the search device outputting the determined installation location information are carried out in the second sub-phase, and
wherein the steps that the sensor device image recording device generates at least one search image, the reference image for the sensor to be located and the search image are transmitted to the image comparison unit, the image comparison unit determining the installation location in the search image or determining that no search image shows the installation location, the transmitting of the result of the image comparison to the search device and the search device determining and outputting the installation information using the image comparison result, are carried out in the second sub-phase.

11. An operating process according to claim 10, wherein, after the first sub-phase, the search device captures a user input as to whether or not the sensor to be located has been located after the first sub-phase, and the second sub-phase is only carried out if the sensor has not been located after the first sub-phase according to the user input.

12. An operating process according to claim 7,
wherein the step that the search device determines the installation location information comprises the step that the image comparison unit compares the at least one search image with each reference image of the sensor to be located and calculates a match indicator for a match between search images and each reference image, and
wherein the step that the search device outputs the installation location information comprises the step that the search device outputs at least one search image which has the greatest match indicator with at least one reference image and/or has a match indicator above a predetermined lower threshold.

13. An operating process according to claim 7,
wherein the generation of at least one search image comprises the additional step that the search device determines a viewing direction in which the image recording device generates the at least one search image,
wherein the step that the search device determines the installation location information comprises the step that the search device determines a path from the location at which the at least one search image has been generated to the installation location of the sensor to be located,
wherein, for determining the path to the installation location, the search device uses the result of the image comparison and the determined viewing directions, and
wherein the step that the search device outputs the installation location information comprises the step that the search device outputs information about the calculated path in at least one form that can be perceived by a human.

14. An operating process according to claim 1,
wherein the or at least one sensor of the sensor arrangement is installed at an installation location that is located in a building with several floors,
wherein the step that the image recording device of the installation device generates at least one reference image of the installation location of the sensor comprises the step that the image recording device generates at least one reference image, which includes an identification of the floor on which the installation location is located.

15. An operating process according to claim 1, wherein the search phase for the sensor to be located comprises the additional steps of:
measuring at least once a current distance between the sensor and the search device, and
outputting an indication for the measured current distance and/or an indication as to whether the measured distance is increasing or decreasing or remains constant.

16. An operating process according to claim 1,
wherein the or at least one sensor comprises a gas measuring device,
wherein the gas measuring device is configured to detect at least one predetermined target gas and/or to measure a concentration of at least one target gas.

17. An operating process according to claim 1,
wherein the or at least one sensor comprises a communication unit,
wherein the communication unit is configured to generate a message and to cause the message to be transmitted to a remote recipient, and
wherein the message comprises information about a measurement result of the sensor.

18. An operating arrangement for operating a sensor arrangement, the sensor arrangement comprises at least one sensor that is configured to measure a physical quantity and/or to check whether the physical quantity lies within a predetermined value range or not, the sensor being configured to be installed at an installation location, thereby becoming an installed sensor of the sensor arrangement, the operating arrangement comprising:
a mobile data-processing installation device, which comprises an image recording device which is configured to generate images;
a mobile data-processing search device;
a central computer, wherein a data connection is established or at least temporarily establishable from the installation device to the central computer; and
a central database, wherein the central computer is configured to create for the or every installed sensor of the sensor arrangement a respective data set in the central database, wherein the data set for the sensor comprises at least one reference image, which is an image that shows the installation location of the installed sensor and has been generated by the installation device,
wherein at least one installed sensor of the sensor arrangement is or can be a sensor to be located,
wherein, for the or every sensor to be located, the operating arrangement is configured to determine each reference image included in the data set for the installed sensor to be located, and, with each determined reference image, to determine installation location information of the sensor to be located and
wherein the search device is configured to output the installation location information in at least one form that can be perceived by a human.

19. An operating arrangement according to claim 18, further comprising a signal-processing image comparison unit,
wherein the search device comprises a search device image recording device which is configured to generate search images,
wherein the image comparison unit is a component of the search device or is at least temporarily in a bidirectional data connection with the search device,
wherein the image comparison unit is configured to compare at least one reference image of the sensor stored in the database with at least one search image generated by the search device image recording device, and determine the installation location of the sensor based on the image comparison or to determine that no search image shows the installation location,
wherein the operating arrangement is configured to detect a predetermined sensor of the sensor arrangement if the installation location is determined in at least one search image and to determine information about the installation location of the sensor, and
wherein the operating arrangement is configured to use the installation location determined by the image comparison to determine the installation location information.

20. A system comprising an operating arrangement according to claim 18, in combination with the sensor arrangement,
wherein the sensor of the sensor arrangement is installed at one installation location, and
wherein the operating arrangement is configured to operate the sensor arrangement.
